(19) United States
(12) Patent Application Publication
(10) Pub. No.: US 2006/0233941 A1
(43) Pub. Date: Oct. 19, 2006

(54) Method of coating a medical device utilizing an ion-based thin film deposition technique, a system for coating a medical device, and a medical device produced by the method

(75) Inventor: Greg Olson, Elk River, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/106,582

(22) Filed: Apr. 15, 2005

(51) Int. Cl. A61L 33/00; B05D 3/00; B05C 5/00

(52) U.S. Cl. 427/2.1; 118/300

(57) ABSTRACT

A method of coating a medical device is provided that includes forming a beam of ions of a coating material and focusing said beam using at least one electrostatic lens. The method also includes arranging the medical device within said beam. In the method, the forming of said beam of the coating material may include aerosolizing a solution of the coating material and evaporating a solvent of the solution. The method may include providing an opposite electrostatic charge to the medical device. The method may include fixturing the medical device to allow the coating material to contact about all of a surface of the medical device. The method may include rotating the medical device about an axis perpendicular to said beam. The method may include moving the medical device through the region of the focus of the at least one electrostatic lens and contacting said beam with a portion of an exposed surface of the medical device. A system for coating a medical device is provided. A medical device having a coating applied by a method is provided.

### FIELD OF THE INVENTION

The present invention relates to medical devices. More particularly, the present invention relates to a method of coating a medical device using an ion-based thin film deposition technique, a system for coating a medical device, and a medical device produced by the method.

### BACKGROUND INFORMATION

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Metal stents may be coated with a polymeric coating that may contain a dissolved and/or suspended bioactive agent. The bioactive agent and the polymeric coating may be dissolved in a solvent mix and spray coated onto the stents. The solvent may then evaporate to leave a dry coating on the stent.

Medical devices may be coated using spray technology. This may entail the use of a two-fluid atomiser, or spray nozzle. The atomiser may be supplied with coating solution and nitrogen gas. The nozzle may be configured so that the coating solution forms a thin film on the pre-filming face of the nozzle, and droplets may then be sheared off the film by the flow of atomising gas.

Spray coating may have a number of limitations. In a spray coating operation, droplet size and droplet velocity may be inextricably linked. It may not be possible to control either of these factors without impacting the other. Additionally, droplet size may only be controlled within a relatively large window due to the gas atomization process. Atomization energy is provided by the nitrogen gas stream. This may result in a high velocity with a correspondingly high energy spray plume, which may significantly increase the difficulty of fixturing stents during the coating process.

Furthermore, the high velocity spray plume produced by two-fluid atomisers may cause a stent to get blown out of alignment on a stent coating fixture. This may lead to difficulty in controlling coat weight, and may lead to coating bare spots due to interaction between a stent and a coating fixture. One approach to counter this issue has been to significantly increase the nozzle-to-stent distance. While this reduces the movement of the stent on the coating fixture, it may result in low coating material efficiencies, perhaps on the order of 1%. A further disadvantage of two-fluid atomisers is that many of the droplets may bounce off the object to be coated, which may further limit the material efficiency. There is therefore a need for reducing coating defects in medical devices.

In the article “Thin organic films by atmospheric-pressure ion deposition”, by Robert Saf, et. al. (Nature Materials, Vol. 3, May 2004), a technique is discussed for depositing thin films of functional organic materials. The article discusses an experimental setup for processing various organic materials into thin structured films under atmospheric pressure. The technique is based on an electrospray process in which microdroplets are initially formed and dried, generating ions that are extracted by electrostatic lenses. Thin structured films are then produced by the deposition of the resulting ion beam onto a moveable target. The technique may offer precise control of film thicknesses and may enable structured depositions.

Each of the references cited herein is incorporated by reference herein for background information.

### BRIEF DESCRIPTION OF THE DRAWINGS

FIG. 1 is a schematic diagram of an exemplary system according to the present invention.

FIG. 2 is a zoomed-in view of an exemplary embodiment of the present invention.

FIG. 3 is a flowchart illustrating an exemplary method according to the present invention.

### DETAILED DESCRIPTION

A method of coating a medical device is provided that includes forming a beam of ions of a coating material and focusing said beam using at least one electrostatic lens. The method also includes arranging the medical device within said beam.

In the method, the medical device may be arranged within a focused region of said beam.

In the method, the forming of said beam of the coating material may include aerosolizing a solution of the coating material and evaporating a solvent of the solution. In the method, the aerosolizing of the solution may include an electrospray process. In the method, the aerosolizing of the solution may include injecting the solution with a capillary having an electrostatic potential. The solution may form the aerosol upon leaving the capillary, and the aerosol may include microdroplets having an electrostatic charge.

The method may include providing an opposite electrostatic charge to the medical device. The electrostatic charge may be oppositely charged of the ions.

In the method, the medical device may include stainless steel, a polymer substrate, a biodegradable material, and/or nitinol and the coating material may include a bioactive agent, an adhesive, and/or a polymer.

In the method, the evaporating of the solvent may include passing heated, dry air through the aerosolized solution.

The method may include transporting the aerosolized solution to the at least one electrostatic lens. In the method, the transporting may be performed by a gas source. In the method, the transporting may be performed by gravity, and the at least one electrostatic lens may be positioned below a source of the ions.

The method may include fixturing the medical device to allow the coating material to contact about all of a surface of the medical device. The method may include rotating the medical device about an axis perpendicular to said beam.

The method may include moving the medical device through the region of the focus of the at least one electrostatic lens and contacting said beam with a portion of an exposed surface of the medical device.

In the method, the medical device may be a stent, a wire, and/or a balloon. The portion of the exposed surface contacted with said beam may include an external pattern of the stent. In the method, said beam contacting the portion of the exposed surface of the medical device may form a layer of the ions of the coating material. In the method, the layer may form a reinforcement band, a geometric pattern, a cone, and/or an expansion profile. The method may include forming another beam of other ions of another coating material and focusing said other beam using the at least one electrostatic lens. The method may also include arranging the medical device in the region of the focus of the at least one electrostatic lens and contacting said other beam with the portion of the exposed surface of the medical device. Said other beam contacting the portion of the exposed surface of the medical device may form another layer of the other ions of the other coating material.

The method may include selecting a desired thickness of the layer of the ions, the selected desired thickness determining at least one of an intensity of said beam and a rate of movement of the medical device through the region of the focus.

In the method, said beam contacting the portion of the exposed surface of the medical device may form a structure of the ions of the coating material, the structure extending away from the exposed surface of the medical device. The structure of the ions of the coating material may form a reinforced band, a pocket, a grid, and/or electrical insulation.

The electrostatic lens may be annular, and said beam may pass through a center of the annular electrostatic lens.

The focusing operation may further include varying a frequency and/or varying an amplitude of an electrostatic force using the electrostatic lens.

The focusing of said beam using the at least one electrostatic lens may further include selecting a size of the ions for the beam and/or selecting a velocity of the ions in the beam.

A system for coating a medical device is provided that includes a source of a coating material and an arrangement for aerosolizing the coating material. The system also includes an air dryer for evaporating a solvent from the aerosolized coating material and at least one electrostatic lens adapted to focus a beam of ions. The system further includes a fixturing arrangement for holding the medical device in a region of a focus of the at least one electrostatic lens.

A medical device having a coating applied by a method is provided. The method includes aerosolizing a solution of the coating material and evaporating a solvent of the solution. The method also includes forming a beam of ions of a coating material and focusing said beam using at least one electrostatic lens. The method further includes arranging the medical device in a region of a focus of the at least one electrostatic lens.

A vapor-based method of thin film deposition of organic materials for stents or other medical devices is provided. In conventional coating applications using organic compounds, the desired material may be dissolved in a solvent and then sprayed, dipped, and/or spin coated. The solvents may then evaporate, or flash off, leaving a coated surface.

The use of an atmospheric-pressure ion deposition process may provide an improved coating method. Elimination of the solvent may be achieved, and a very controllable process for the material deposition, including thickness, location, and/or rate, may be achieved. This process may be performed under atmospheric pressure, rather than a vacuum, as may be required for some variants of current electrostatic processes.

Ionized molecules of the desired organic material are generated and focused by electrostatic lenses onto a substrate. One difference between this process and other electrostatic processes is that conventional electrostatic processes create a charged microdroplet and then directly deposit this droplet onto a substrate. In contrast, the present invention provides a process that dries the microdroplet into a charged particle, which is then directed, under control, by an electrostatic lens onto a medical device.

The process provides the ability to create thin structured films of organic materials under atmospheric pressure. The technique is based on an electrospray process, but in this situation the microdroplets are initially formed and dried, and the generated ions are extracted by electrostatic lenses.

Thin structured films may then be produced by the deposition of the resulting ion beam onto a moveable target. The technique offers several interesting features, including precise control of film thickness. It may also be possible to form structured deposits and/or thin films with varying chemical compositions. The technique may be utilized to arrange organic coatings (for example, polystyrene or insulin) onto stents or other medical devices.

Materials such as polystyrene, polymethylmethacrylate, angiotensin, and insulin may be compatible with the disclosed method.

This process may avoid the limits of other solution-based techniques (for example, spincoating or inkjet spraying) in which the materials that are deposited may also react with the solvent used in the application technique, either in the coating or in the application of the next layer of the coating.

The method may enable a very controlled deposition of organic materials into different geometric shapes and thicknesses. It may also be used for bulk coating of a component.

Removal of the solvent from the coating processes may achieve significant safety and/or manufacturing cost advantages.

The technique may provide an improved yield, and 50% to 95% of the coating material injected may be transferred to the target.

The technique may improve surface roughness, and may provide a smooth, coated surface. This process may also improve the morphology of the films. The technique may reduce and/or eliminate holes or other defects, and may reduce cratered areas caused by the impact of droplets. Furthermore, since the particles are dry at impact, there may be little or no force exerted on the surface by a drying process.

This technique may be used to coat stents with polymer or polymer/drug combinations. The layers may be applied thinner than conventional methods. The stacking of thin layers may also be possible, and the stacks may be of either homogeneous or heterogeneous construction. Although the process has been focused on organic coatings in the nanometer ranges (70-100 nm), thicker sections may be derived from a higher input rate of material, and/or a slower movement of the stent or medical device.

Conventional SIBS coatings may be in the 20 micron range, but the thickness could be thinner and still accomplish the required release kinetics. Thinning the coating may make it more flexible, which may be beneficial since the coating needs to conform to deformations of the stent body without cracking and/or peeling.

The technique may be used to develop polymer/biomaterials structures on stents, wires and balloons. An example of this would be to create reinforcement bands, geometries on balloon bodies, cones for strength increase or wear increase, or to create a custom pressure or diameter expansion profile of a balloon.

Stents may be coated selectively (e.g., on only one side) using this technique. For instance, the vessel contact side may be coated while the interior of the stent remains uncoated. The increased controllability or layering of materials (including bioabsorbables) may allow a broader range of custom drug release profiles. Geometries could be constructed on the surface of stents, like pockets, grids, etc. for holding different concentrations of drugs, or different drugs themselves. The application control may allow application of different materials in different layers, longitudinally in distinct bands, and/or following other geometric configurations, for example varying thicknesses. This layering application control in three dimensions may be used in conjunction with the stent geometries to optimize the release kinetics at the vessel wall.

This technique may also be used to apply adhesives to join components. For example, very thin layers may be produced that could be used to eliminate and/or reduce the ‘bond gap’ (i.e., the space between two concentric bodies) needed to wick-in adhesive materials. The technique may also have applications in which a very thin tie layer of a joining material is provided, which may be used to join two incompatible materials.

This technique could also be applied to coatings applications for balloon catheters and guidewires for increased lubriciousness. An extremely thin but effective coating layer may be provided by the technique.

The process may be compatible with a wide range of compounds, including polystyrene, polymethylmethacrylate, angiotensin (a compound that causes muscle cell contraction) and insulin (a polypeptide hormone). Degradation reactions of these materials may be avoided by using this process. Therefore, materials derived from the ethylene molecule (including polypropylene, polyethylene, polystyrene) may be successfully processed using the technique. Additionally, polymers derived from the acetylene molecule (including polytetrafluoroethylene and other teflon derivatives) may also be successfully processed using the technique.

This process may be used to apply polymer materials to form an electrical insulation for theraputic or diagnostic devices, for instance electrophysiology products using electrical conduits. These devices may use either jacketed wires or alternating layers of polymer tubing, and these wall thickness may be dependent on the polymer materials. Using the provided technique may reduce wall thicknesses.

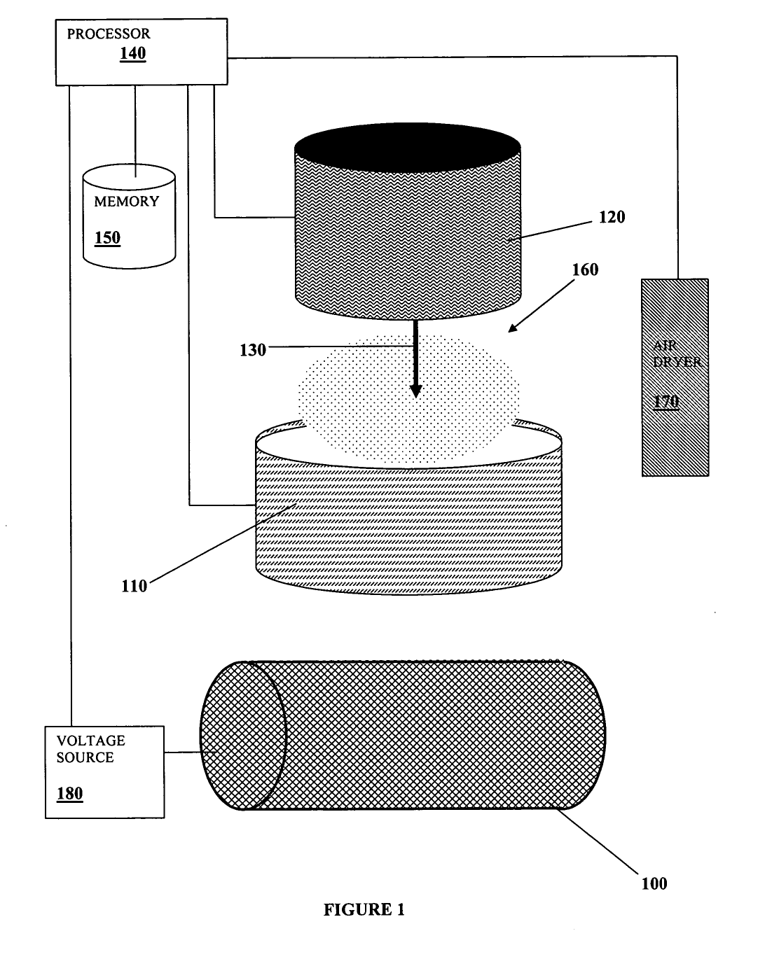

FIG. 1 is a schematic diagram of an exemplary system according to the present invention. Stent 100 is shown positioned below electrostatic lens 110. Electrostatic lens 110 is positioned below coating source 120. Electrostatic lens 110 includes an annular shaped lens able to focus an ion beam. Coating source 120 provides aerosolized cloud 160, composed of aerosolized particles of coating material. Aerosolized cloud 160 is directed in the direction of arrow 130 towards electrostatic lens 110. Coating source 120 may be an electrospray system or any other method of aersolizing coating material. If aerosolized cloud 160 includes solvent, air dryer 170 may be utilized to evaporate the solvent by flowing hot, dry gas over and/or through aerosolized cloud 160. Coating source 120 may impart an electrostatic charge to aerosolized cloud 160, which may maintain the electrostatic charge after the removal of the solvent, if any. Aerosolized cloud 160 may consist of microparticles of coating material having an electrostatic charge upon entering electrostatic lens 110. The ions in aerosolized cloud 160 may be influenced by the electromagnetic field produced by electrostatic lens 110. Electrostatic lens 110 may accelerate and/or focus the ions in aerosolized cloud 160, and may direct the ions towards stent 100. Stent 100 may have an electrostatic charge opposite the charge of ions in order to assist in attracting the beam of ions. The electrostatic charge on stent 100 may be provided by voltage source 180. Voltage source 180 connected to stent 100 may impart an electric potential that provides a charge to stent 100 that is opposite to the charge of the ions. The ions may produce a uniform coating on stent 100. Processor 140 coupled to memory 150 may contain and/or execute instructions for operating coating source 120, electrostatic lens 110, air dryer 170, and/or voltage source 180.

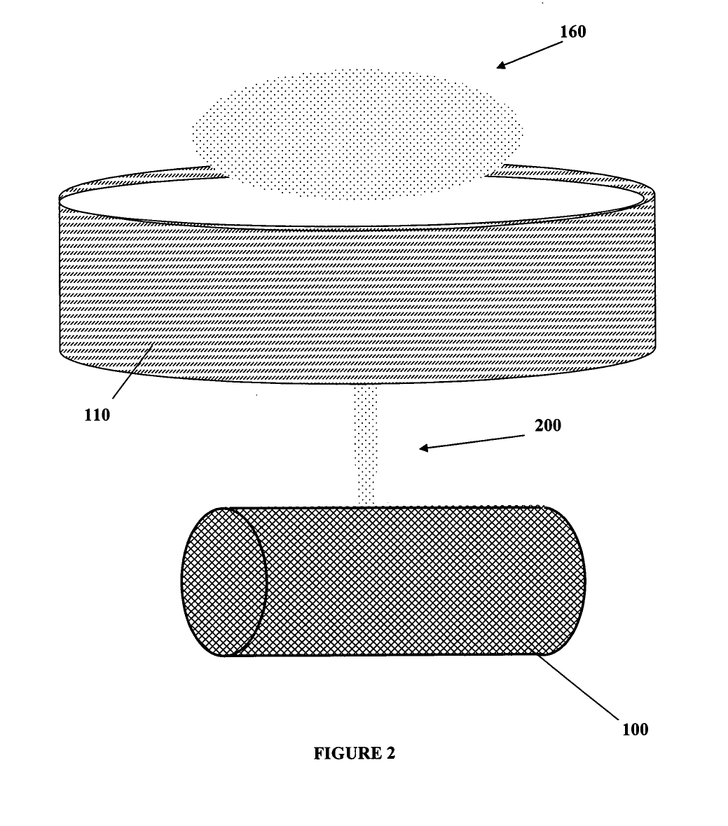

FIG. 2 is a zoomed-in view of an exemplary embodiment of electrostatic lens 110. Aerosolized cloud 160 may be aerosolized coating material that has been dried to remove any solvent and may be composed of ions. Aerosolized cloud 160 may enter electrostatic lens 110, which may then focus and/or accelerate the ions in aerosolized cloud 160. The ions of aerosolized cloud 160 may be focused into ion beam 200, which may be directed at the externally exposed surfaces of stent 100. Stent 100 may be fixtured on fixture 210, which may be adapted to move stent 100 in any direction, including perpendicular to ion beam 200 and parallel to ion beam 200. Additionally, fixture 210 may be adapted to rotate stent 100, for instance around axis 220 in the direction of rotational arrow 230. Processor 140 coupled to memory 150 may contain and/or execute instructions for operating fixture 210 as well as electrostatic lens 110.

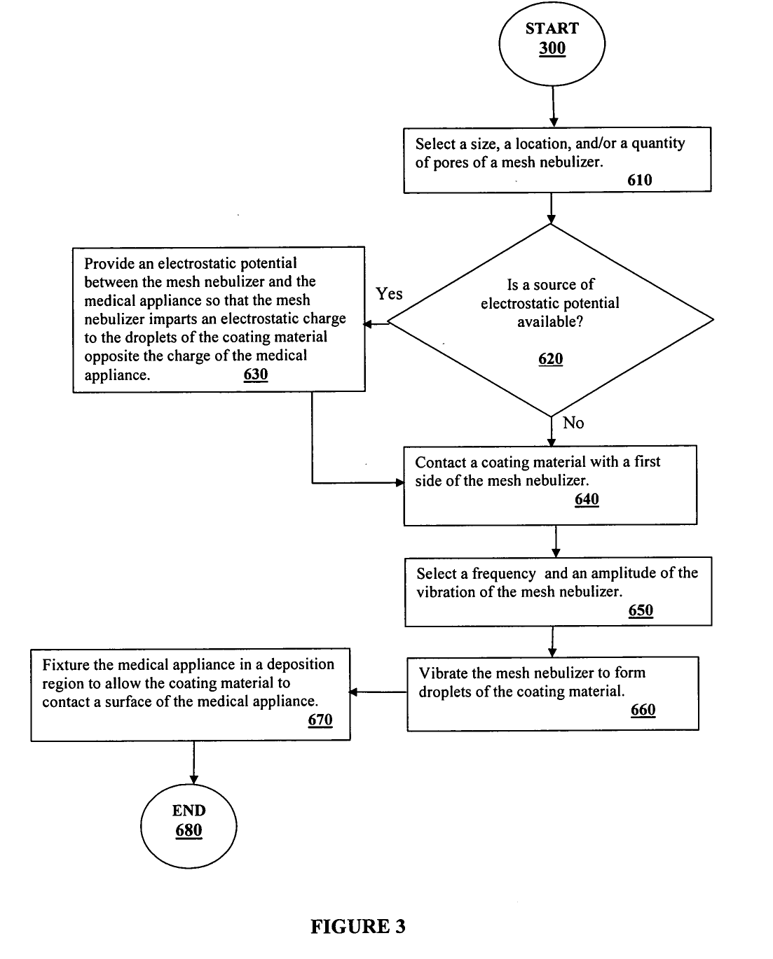

FIG. 3 is a flowchart illustrating an exemplary method according to the present invention. The flow in FIG. 3 starts in start circle 300 and proceeds to action 310, which indicates to aerosolize a solution of the coating material. From action 310, the flow proceeds to action 320, which indicates to evaporate a solvent of the solution. From action 320, the flow proceeds to action 330, which indicates to form a beam of ions of a coating material. From action 330, the flow proceeds to action 340, which indicates to focus said beam using at least one electrostatic lens. From action 340, the flow proceeds to action 350, which indicates to arrange the medical device in a region of a focus of the at least one electrostatic lens. From action 350, the flow proceeds to end circle 360.

As used herein, the term “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

What is claimed is:

1. A method of coating a medical device, comprising:
forming a beam of ions of a coating material;
focusing said beam using at least one electrostatic lens; and
arranging the medical device within said beam.

2. The method of claim 1, wherein the medical device is arranged within a focused region of said beam.

3. The method of claim 1, wherein the forming of said beam of the coating material comprises:
aerosolizing a solution of the coating material; and
evaporating a solvent of the solution.

4. The method of claim 3, wherein the aerosolizing of the solution further comprises injecting the solution with a capillary having an electrostatic potential, the solution forming the aerosol upon leaving the capillary, the aerosol including microdroplets having an electrostatic charge.

5. The method of claim 3, wherein the evaporating of the solvent further comprises passing heated, dry air through the aerosolized solution.

6. The method of claim 1, further comprising providing an electrostatic charge to the medical device, the electrostatic charge being oppositely charged of the ions.

7. The method of claim 1, wherein:
the medical device comprises at least one of stainless steel, a polymer substrate, a biodegradable material, and nitinol; and
the coating material comprises at least one of a bioactive agent, an adhesive, and a polymer.

8. The method of claim 1, further comprising fixturing the medical device to allow the coating material to contact about all of a surface of the medical device.

9. The method of claim 8, further comprising rotating the medical device about an axis perpendicular to said beam.

10. The method of claim 1, further comprising:
moving the medical device through the region of the focus of the at least one electrostatic lens; and
contacting said beam with a portion of an exposed surface of the medical device.

11. The method of claim 10, wherein:
the medical device is at least one of a stent, a wire, and a balloon; and
the portion of the exposed surface contacted with said beam includes an external pattern of the medical device.

12. The method of claim 10, wherein said beam contacting the portion of the exposed surface of the medical device forms a layer of the ions of the coating material.

13. The method of claim 12, wherein the layer forms at least one of a reinforcement band, a geometric pattern, a cone, and an expansion profile.

14. The method of claim 12, further comprising:
forming another beam of other ions of another coating material;
focusing said other beam using the at least one electrostatic lens; and
arranging the medical device in the region of the focus of the at least one electrostatic lens; and
contacting said other beam with the portion of the exposed surface of the medical device;
wherein said other beam contacting the portion of the exposed surface of the medical device forms another layer of the other ions of the other coating material.

15. The method of claim 12, further comprising selecting a desired thickness of the layer of the ions, the selected desired thickness determining at least one of an intensity of said beam and a rate of movement of the medical device through the region of the focus.

16. The method of claim 10, wherein said beam contacting the portion of the exposed surface of the medical device forms a structure of the ions of the coating material, the structure extending away from the exposed surface of the medical device.

17. The method of claim 16, wherein the structure of the ions of the coating material forms at least one of a reinforced band, a pocket, a grid, and electrical insulation.

18. The method of claim 1, wherein the at least one electrostatic lens is annular, said beam passing through a center of the at least one annular electrostatic lens.

19. The method of claim 1, wherein the focusing operation further includes at least one of varying a frequency and varying an amplitude of an electrostatic force using the at least one electrostatic lens.

20. The method of claim 1, wherein the focusing of said beam using the at least one electrostatic lens further includes at least one of:
selecting a size of the ions for the beam; and
selecting a velocity of the ions in the beam.

21. A system for coating a medical device, comprising:
a source of a coating material;
an arrangement for aerosolizing the coating material;
an air dryer for evaporating a solvent from the aerosolized coating material;
at least one electrostatic lens adapted to focus a beam of ions; and
a fixturing arrangement for holding the medical device in a region of a focus of the at least one electrostatic lens.

22. A medical device having a coating applied by a method, the method comprising:
aerosolizing a solution of the coating material;
evaporating a solvent of the solution;
forming a beam of ions of a coating material;
focusing said beam using at least one electrostatic lens; and
arranging the medical device in a region of a focus of the at least one electrostatic lens.
